(19) United States
(12) Patent Application Publication
(10) Pub. No.: US 2010/0137953 A1
(43) Pub. Date: Jun. 3, 2010

(54) DEVICE FOR COOLING

(76) Inventor: Gabriele Stein, Elchingen (DE)

Correspondence Address: JACOBSON HOLMAN PLLC, 400 SEVENTH STREET N.W., SUITE 600, WASHINGTON, DC, 20004, US

(21) Appl. No.: 12/452,992

(22) PCT Filed: Aug. 1, 2008
(86) PCT No.: PCT/EP2008/006360

§ 371 Date: Feb. 1, 2010

(30) Foreign Application Priority Data
Aug. 1, 2007 (DE) 10-2007-036-496-4

(51) Int. Cl. A61F 7/02; A61F 7/00

(52) U.S. Cl. 607/112; 607/96

(57) ABSTRACT

The invention relates to a cooling device, especially for cooling bodies or body parts of humans or animals, comprising at least one supporting material that contains superabsorber (SAP) which can absorb and re-dissipate moisture or liquid. The supporting material is wrapped in a covering fabric that has at least one moisture-permeable or air-permeable and steam-permeable zone. The invention is characterized in that at least one measuring instrument and/or sensor for measuring physical measuring values is provided on or in the device.

The invention refers to a device for cooling, in particular for cooling bodies or body parts of a human or an animal, comprising at least one supporting material with super absorber (SAP) which is able to absorb, bind and release again moisture wherein a cooling effect occurs by evaporating when the moistures is released, wherein the supporting material is wrapped in a cover fabric and the cover fabric has at least one moisture-permeable or air- and steam-permeable area.

In the state of the art the above-described devices are known for cooling or cooling systems which work, for example, with water cooling. Here devices of this kind are equipped as mats or as vests with a pipe system through which water is pumped. This water has to be cooled in an electric apparatus. The above-mentioned systems have the disadvantage that they are rather expensive because of the required apparatus and can mostly used only stationary.

Furthermore it is known in the state of the art to cool bodies by offering infusions with cooled liquid. This method depends on apparatus, is very expensive and therefore only suitable for stationary applications.

Furthermore it is known in the state of the art to guide cooled air in suitably equipped vests and mats. Here also the effort is high and the apparatus expensive. Furthermore, here also the radius of movement, for example of a patient, is clearly restricted.

In the state of the art furthermore cooling systems have been known working based on a cooling with ice. These cooling systems have the disadvantage that they may lead, as a rule, to a vasoconstriction of the blood vessels and thus to risks in the medical treatment which cannot be calculated.

Furthermore so-called gel accumulators, chambers filled with gel, are known wherein this gel is generated by filling in water in pockets with super absorber crystals. These gel accumulators, whether in synthetics or textiles, have also to be put in the refrigerator or in a freezer compartment before they can be used. Besides, the disadvantage here is a rather high weight. This puts additional stress on the patient.

Furthermore cooling systems are known which are a development of the accumulator principle. These solutions work with materials of higher thermal conductibility. Here it is also necessary to activate the corresponding device with water and to cool it in a cooling apparatus accordingly.

Furthermore it is known to use so-called PCM (Phase Change Materials). These systems contain packages with salts or other materials which absorb heat at higher temperatures by melting and cool at the same time. A very short effective cooling time, high weight and expensive activating measurements are the disadvantages of this solution.

Also cooling systems with super absorber crystals have become known. Most of the cooling systems containing super absorber crystals which absorb water through contact with it and thus get a gel-like consistency belong to the further above-described category of gel accumulators, as the cooling effect by evaporating is very small. Therefore here also cooling in the freezer is recommended in order to get a sufficient cooling effect.

Furthermore devices or cooling systems have become known working with evaporative cooling without super absorber. These systems, however, can only absorb a small volume of water as the water has only a limited cooling effect. Besides these systems are characterized disadvantageously in that there is a very small volume of water leading again to a short cooling period.

All apparatus-depending systems, as described above, have the disadvantage that they can only be used with very large effort and thus very expensive in the medical field, and have the disadvantage that the patients are very limited in their freedom of movement. Additionally the apparatus-dependent or even the so-called variable systems cannot be controlled and are already for this reason not suitable for medical applications.

In the field of thermal protection, for example in fire fighting, it is known to provide a device of a supporting fleece coated with super absorber (SAP). Furthermore, this supporting fleece is provided in an at least partially moisture-permeable or air- or steam-permeable cover. By absorbing water and its evaporating starting by the thermal influence from the outside a cooling of the device is the result so that it can be used for fire protection. It is the disadvantage of this known device that the device is not suitable for a medical cooling application in this form. The reason for this is also that the cooling cannot be measured and therefore not controlled.

Coming from the before-described solutions of the state of the art it is an object of the invention to suggest a device for cooling bodies or body parts of a human or an animal which does not have anymore the disadvantages known from the state of the art.

The problem of the invention is solved by a device for cooling, in particular for cooling bodies or body parts of a human or an animal as described in the beginning which is characterized in that at least one measuring instrument and/or sensor for measuring physical measuring values is provided at or in the device.

The invention takes into consideration that medical cooling systems have to be measurable and controllable as the temperature of the body has a direct influence on the vital body functions. The cardio-vascular system, for example, is directly connected with the body and skin temperature and regulates them in order to keep up vital functions. If the organism is cooled to low (hypothermia) or not cooled enough and therefore overheated severe or life-threatening situations may be the result. Therefore a control and/or monitoring of the cooling is almost always compulsory in the medical field. With the device containing at least one measuring instrument and/or sensor for measuring physical measuring values the invention provides such a solution which can be employed excellently in the medical field.

Referring to the knowledge that evaporating heat also occurs when, for example, it is provided by the body temperature of a human or an animal the invention now suggests a device which provides at or in the device itself a measuring instrument or a sensor for measuring physical measuring values. By means of this the temperature of the device and/or the person using it or patient can be determined very accurately so that also, for example, by adding moisture purposefully the cooling temperature can be increased, or no moisture is added to the device so that the temperature settles again to a normal value. It is a particular advantage of the device that by means of the arrangement of measuring instruments or sensors, preferably in such a way that they are provided closely to the body or directly at the skin or surface of the patient, the physical data of these patients can be determined very easily so that also here very easily an influence can be exerted purposefully.

Furthermore it is a big advantage of this device that it is possible now to provide parts of the body or the whole body altogether with a device, and the patient or user of this device still has sufficient freedom of movement so that he or she can move freely. In the variable solutions of the state of the art this was completely impossible. Now it is not necessary anymore either to provide special devices for cooling, for example for water cooling or air cooling such as in the solutions of the state of the art. It is no more necessary either to provide cooling aggregates or deep-cooling aggregates, as it was necessary in the state of the art in order to bring the devices to a low temperature beforehand.

The invention here comprises not only medical applications, that is cases where a patient is monitored and has to be provided by suitable cooling. The use of a measuring instrument at the device according to the invention is also an advantage for auto-monitoring the device. It is an advantage of the invention that it can be activated by filling the supporting material with liquid, as for example water, that means then it provides a suitable cooling potential. This cooling potential is provided permanently, a suitably active support, as for example by means of a cooling aggregator and so on, is not necessary. The cooling effect here or the cooling efficiency depends on how the liquid bound in the super absorber escapes again from the super absorber, and the water or moisture steam generated with it disappears from the device. It has namely been observed that the cooling efficiency is accordingly higher when the physical processes decisive for the cooling effect are supported. For that purpose a sufficient gradient of the steam partial pressure in has to be provided, on the one hand, in the area of the device, and also a sufficient temperature gradient, on the other hand, at or in the device or the supporting material, which is, if necessary, a supporting fleece. If corresponding physical parameters or difference measurements become to low the cooling efficiency decreases accordingly, although the device itself or the supporting material is charged sufficiently with liquid and would still provide a suitable cooling effect.

By means of the measuring instruments suggested by the invention therefore not only a reliable system, which is characterized in that the cooling effect is provided at a physiologically tolerable temperature level (for example between 15° C. and 30° C., icing is not intended) is suggested for the therapy of patients, for example in accident or other life-threatening situations, and which additionally does without additional extern and expensive cooling aggregates and cooling machines. The suggestion according to the invention, however, also offers a self-sufficient control monitoring for the cooling efficiency of the device if it is used, for example, as piece of clothing in heated working areas.

The invention is also characterized by a very low weight, and can be kept or set in particular for a very long period of time to a suitable temperature. This can be reached, for example, also by adding again purposefully moisture in certain intervals in order to reach the cooling effect again. For that it may be enough, for example, when a moisture sensor is integrated additionally to the measurement of the measuring values of the patient in the device and because of this display then moisture can be added in order to increase the cooling effect again. The cooling effect occurs in particular because the supporting fleece carries super absorber which is coated there or otherwise applied. In contrast to the solutions known in the state of the art with crystals or gel as super absorber which is sewed in pockets this solution with a coated super absorber on the supporting fleece is able to absorb very fast moisture and also to release it again without the disadvantageous effects of escaping gel. In the state of the art this occurred frequently so that the designed solutions could not be applied for medical purposes.

Furthermore also dripping as well as wet clothing is prevented as the cover fabric or the cover of the device is designed in such a way that, on the one hand, in one direction moisture may enter, on the other hand, however, no moisture gets through, for example, to the skin or body of the patient so that soaking cannot occur. According to the invention the cover fabric is steam-permeable, at least in partial zones, so that the moisture can pass from the outside to the inside to the supporting fleece, and, on the other hand, steam diffusion is also possible to the outside. The surface on the side of the cover fabric facing the patient, however, can be water-impermeable according to an embodiment of the invention so that here no soaking can occur, as it occurred inconveniently in the state of the art.

In contrast to all solutions known in the state of the art, be it cooling systems with water cooling outside the body or by infusion within the body, be it cooling systems with air cooling, cooling with ice, cooling with gel accumulators or by developed accumulators with higher conductibility, or even cooling systems with the so-called Phase-Change-Materials (PCM), the solution according to the invention is characterized in that it is very easy, that the patient can carry it on the body without disadvantageous effects such as soaking or hypothermia. Furthermore the solution according to the state of the art is characterized in that the temperature can be, for example, adjusted purposefully and controlled to a certain range which has not been possible with any of the systems of the state of the art. Furthermore, the solution is characterized in that by coating a supporting fleece with super absorber now the moisture absorption can take place very fast so that, for example, under the faucet or the shower it can be moisturized in a very short effective time, and then by evaporations, caused by body temperature, a cooling effect is reached which is ideal for medical purposes, as in particular also by employing measuring instruments and/or sensors which, of course, can be coupled with suitable monitoring systems, it can be adjusted to the respective case of application without any problems.

Of course also the cover fabric itself or parts of the cover fabric can carry out the data transmission if for the production of the fabric a cable-like textile thread is used with the capability of screening radiation or transmitting data, serving as power source or substituting sensors, and which can be used as one. This thread or parts made from it can also be used as link for transmitting energy and/or data or be connected with joints.

For cooling in the medical field, as they have been known to be carried out so far the device according to the invention is excellently suitable. Thus it is also possible according to the invention now to carry out cooling for blunt injuries, sprainings or bruises, which so far have been treated with ice, ice accumulators, gel pillows or the like, with the device according to the invention. The risks pointed out as warning by specialized doctors and physiotherapists of hypothermia of the areas by the ice and in particular also because of a possible constriction of the vessels are prevented completely according to the invention. This goes not only for injuries of the respective persons but also for injuries of animals which also in the state of the art have been treated with ice, frozen gel pillows, accumulators or with cold water, compresses or the like.

Also the cooling of cardio-vascular troubles, which could be treated so far only rarely or unsatisfying in the state of the art, can now be solved elegantly with the device according to the invention. Thus it is now possible, in particular with older patients with cardio-vascular ailments or also with animals, such as for example older dogs with cardio-vascular ailments, to carry out a medically controlled cooling, in particular even with high outside temperatures. Just in this field it is suitable to offer the climatic control of the body in the form of cooling pieces of clothing which should, of course, because off the already weaker cardio-vascular system have less weight and no ice temperatures. By applying such a piece of clothing or a blanket according to the invention no water drops or gel rests occur which could get to the body surface of the affected person. In this respect also the cooling when cardio-vascular troubles exist is an ideal field of application of the device according to the invention. The disadvantages known in the state of the art are completely avoided. As it is important just during cooling application in the medical field or during cooling applications in this area to monitor the temperature to get the medical advantage without uncontrollable side effects, this is guaranteed now by the chances of the device according to the invention by the invention for improving the state of the art.

With some medical indications it may be useful to cool the patient during a certain period of time more than normally. That means that the body temperature is lowered purposefully to an actually health-damaging temperature of 33 to 34° C. or less. Thus side effects such as hypothermia or infections are accepted as the more severe emergency can only treated better by this. This is possible, for example just with patients with heart attacks who are undercooled purposefully as by means of that, according to researches, subsequent damages can be minimized. Exactly in this case control and monitoring of the cooling as well as also of the medical parameters is urgently required.

Also this way of cooling can be guaranteed by means of the device according to the invention. In this case the device can be stored, for example in the form of a blanket, in the refrigerator or in the freezer (ahead activated with water), until the medically desired temperature has been reached. The super absorber fleece holds the temperature longer than this would be the case with water or ice, so that here also an advantage occurs in the use. The weight is lower than that of ice packs and the effect more enduring. Additionally, the flexibility and the possibility of adapting to the body remains which makes the application at the patient handy and convenient. The body functions and the temperature are monitored. As no water or gel escapes all other necessary links to the patient, such as, for example, attached electrodes, keep their effect which can lead to problems such as system failures which may be life-threatening when ice water or similar measurements of the state of the art have been used. Additionally, such products according to the invention can be stored and/or carried readily confectioned with water and packed sterile in ice boxes of ambulance cars or other suitable places and also with endangered persons at home or underway as emergency product, so that they can be used immediately in an emergency of a heart attack. As in these cases the shortness of time may save lives or prevent subsequent complications the invention serves for the health of such affected persons.

It is known in the field of medicine to treat patients with multiple sclerosis by cooling. The heat sensitivity characterized by the Uhthoff phenomenon of about 80% of the affected persons with multiple sclerosis (MS) leads to stronger symptoms as well as to exhaustion with higher temperatures. These symptoms can be reversed, as it is known by cooling the body. It is even known that by cooling the body the undesired symptoms do not occur in the first place. As the affected persons as a rule are already very restricted in their freedom of movements, however, want to take part in social life, cooling clothing systems should be light and for health reasons not have ice temperatures. Here also pieces of clothing should not allow water drops on the skin or gel rests to occur. All these negative symptoms, as they are known in the state of the art, are avoided completely by the invention. In particular, now monitoring the temperature is possible according to the invention.

The invention can be used excellently by cooling, for example, patients with heart attacks with suitable pieces or partial pieces of clothing. In numerous researches and publications it has been stated that the survival rate of patients with a heart attack is significantly higher when the body of the affected person is cooled below the usual body temperature. A solution which would in fact take place through the emergency treatment from the place of collecting the patient until the hospital, that means even during the transport, or does not interrupt the cold chain during transport to the hospital is not a given until today in medical routine. The invention now provides a device which could achieve that. The disadvantages such as moisture, uncontrolled ice temperature, uncontrolled cooling temperature by accumulators, escaping gel and so on is all avoided completely by the invention. It is even possible to adjust purposefully temperatures so that by means of the invention a system is provided at the same time which can be used in the medical field for humans as well as for animals. This is also the case because an uncontrolled or unadjusted cooling which may be life-threatening is now completely excluded.

There are, of course, other cooling applications in the field of medicine to which the system according to the invention or the device according to the invention can be transferred without any problems. It is only important that the cooling used so far uncontrolled with the exception of cooling-medical apparatus and all connected disadvantages are removed and a professional, device-independent cooling system or a device for medical indications is created.

In a preferred embodiment of the invention it is provided that by means of the measuring instrument or the sensor a determining and monitoring of the cooling efficiency is carried out. It has been described that by releasing moisture by evaporating a cooling effect occurs. This evaporation enthalpy is taken from the surroundings (e.g. the body) and this is cooled accordingly. By means of the SAP charged supporting material a kind of artificial sweating is effected which leads to a corresponding removing of energy from the body. The user wants to reach a suitable efficiency by using the device according to the invention, he or she wants to experience a certain cooling efficiency. As a separate device application for removing the steam from the device is deleted, in a modification according to the invention it may happen that the user does not feel a cooling efficiency anymore although the device still may have cooling effect, that means is charged with suitable liquid. It is an essential advantage of the invention that the measuring instruments or sensors suggested according to the invention are used in a modification according to the invention to determine or monitor the cooling efficiency. For that purpose the physical measuring values measured by the measuring instruments or sensors, such as for example temperature, temperature gradient, steam partial pressure, relative air humidity, charging condition of the supporting material with SAP (Super Absorber Polymers) and so on are collected in a suitable way and processed, in order to make a conclusion for the cooling efficiency, and thus to generate a hint for the user if the cooling efficiency felt by the user is lowered under a corresponding threshold value. Although the device as passive system, basing for example on convection, may show a release of steam alternating during the time which acts directly on the cooling efficiency, by means of the invention an optimum use is reached.

According to an advantageous development of the invention the device is characterized in that as measuring instrument or sensor at least one thermometer or thermal sensor is provided. This measuring instrument or sensor determines preferably the temperature of the patient or user and can give hints whether the cooling is sufficient or whether further cooling is necessary. Because of this information then moisture can be added to the device so that a higher cooling effect occurs. Or the effect of the information about the measurement is that the cooling is sufficient. In this very uncomplicated way it is possible to carry out, first of all, a cooling suitably and, secondly, to monitor also the cooling accordingly. It is possible here that the measuring instrument displays the temperature directly or the measuring instrument or sensor transmit the collected information to an evaluating unit. Here again it is possible that the evaluating unit is located directly at the device or that a central evaluating unit, selected for example via radio, collects the data and evaluates them accordingly. An evaluation and/or transmission can here be done by a personal computer, a mobile phone, a mini computer (PDA—Personal Digital Assistant).

Thus an examination within a restricted space defined by the range of the transmission is possible. With up-to-date telemedical apparatus monitoring is even possible over long distances to the doctor, hospital or the rescue headquarters.

In different embodiments according to the invention different suggestions are made for the design of the measuring instrument or the sensor. They serve in particular for defining the cooling efficiency. Besides the use of a thermometer, as already described, for example the use of temperature difference measurement is suggested where, for example, with regard to two moments or with regard to two places the temperature difference is determined. In thermodynamic respect it is clear that a temperature gradient is a way of heat transport and thus serves for determining the cooling efficiency. Alternatively according to the invention also the use of an air humidity sensor or am air humidity difference measurement is suggested. By determining the relative air humidity the water steam partial pressure can be concluded. In a modification according to the invention a measuring instrument is suggested which may directly measure the steam partial pressure and serves as a measurement for the determination of the cooling efficiency.

By means of resistance or capacitive measurement arrangements in the device, in particular in the area of the supporting material equipped with SAP it is possible in another modification according to the invention to define a liquid charging sensor so that by determining the liquid level and its change directly the cooling efficiency, which is actually a function of the mass loss of liquid in the super absorber polymer, can be concluded. The suggestion of a capacitive design of the liquid charging sensor is only a modification here. Here the fact is used that the capacity of a super absorber polymer filled with liquid changes and is thus a measurement for the charging condition.

It is clear that also combined measuring instruments, such as for example a climate measuring apparatus, can be employed according to the invention where, for example, several physical parameters (temperature, relative air humidity and so on) are measured.

All these parameters can be used for determining the cooling efficiency and serve for it.

Cleverly it is suggested that the cooling efficiency is determined by measuring the difference of the measuring values either with regard to the place of measurement and/or the moment of measurement. It has been observed, for example, that the accumulation of steam in the device (for example in a vest) is characterized by water steam partial pressure or air humidity, which does not change anymore with respect to time or is spatially constant. In order to monitor the cooling efficiency it is therefore convenient to observe these gradients either with respect to time or to place and to base the evaluation on them.

In a preferred embodiment of the invention it is provided that the thermometer is designed as liquid crystal thermometer. A thermometer of this type uses the thermometer fluorescence effect. Here reversible temperature stickers of liquid crystals are known which have multi-coloured analog displays. The advantage of the use of this thermometers is, in particular, their economic price, their sufficient accuracy and also their use without any problems in a device which, according to its design, comes into contact with liquid as, for example, it is wetted, and so on. A suitable device equipped with this thermometer can even be washed without any problems.

Cleverly then at the device two or more thermometers equipped accordingly as liquid crystal thermometer are used, and the user determines himself or herself by sequential reading of the respective temperatures the temperature difference and concludes thus the still existing cooling efficiency.

In a development according to the invention it is provided that for determining the cooling efficiency the measuring instrument or the sensor is equipped or connected with an evaluating unit. During the determination of the cooling efficiency very different parameters are received. Of course, these parameters depend on the measured physical measuring values. It has been found here that for the different measuring values there are also corresponding algorithms or scenarios in order to determine or detect a cooling efficiency. Exemplarily it has been found that a permanent steam partial pressure within the device seen locally between a measuring point quite close to the body surface and a second measuring point, for example, within the carrier material or at its end opposite the body, is a good base for judging the cooling efficiency. If the gradient has disappeared, that means everywhere there is the same water steam partial pressure, then there is no more cooling effect the felt cooling efficiency is accordingly low. This is determined by means of the evaluating unit according to the modification of the invention.

Furthermore a display for the cooling efficiency is provided which displays the information determined by the evaluating unit. The display here aims for example in a first modification to display only information for the cooling efficiency which then informs the user accordingly to change, for example, the device or to put it off for a short time and then to put it on again, for example, to solve a corresponding heat or steam accumulation.

In a very simple modification here the display consists of two lights with different colors where, for example, a green light shows good cooling efficiency and a red light shows poor cooling efficiency, and requests suitable measurements. The display can be here become complicated in any way. Additionally in the display, of course, also other physical or physiological values can be shown.

In another embodiment according to the invention it is provided that the measuring instrument or the sensor is arranged at or in the supporting material. Generally, it has to be pointed out that the measuring instrument or the sensor is not only characterized by an apparatus for collecting a measuring value, but, of course, the measuring instrument or the sensor comprises several measuring points, and is thus, for example, provided at different positions at or in the supporting material, or at or in the device. Thus it is, for example, also provided that the measuring instrument or the sensor is arranged on the side of the supporting material facing and/or opposite the human or the animal. Often just the gradient with regard to the side facing the body and opposite is interesting, and the measuring arrangement is accordingly constructed and arranged.

It is a big advantage to provide a protective pocket or protective flap at the device for the measuring instrument or the sensor. The protective pocket is, for example, of water-impermeable material and protects the possibly sensitive measuring arrangement, sensor and, if necessary, the evaluating unit against liquid. But also a protection against parasitic heat sources, such as, for example, insolation which is undesired can be a massive advantage for the accuracy of the measurement by an arrangement of the measuring instrument (thermometer) behind a suitably well insulated protective flap at the device.

In a preferred embodiment of the device according to the invention it is provided that the measuring instrument or the sensor is arranged on the side of the cover fabric or cover facing the body, in particular, preferably in such a way that the measuring instrument or the sensor gets direct body or skin contact when the device has been put on. By this process it is possible to measure the body temperature or other values directly at the patient or user or even at the animal carrying the device. This increases the accuracy considerable. In contrast to the devices known in the state of the art which—as already mentioned—could be only used for cooling purposes for fire protection tasks or heat work, the thermometer according to this embodiment is directly on the side which gets in contact with the patient, so that here a direct body or skin contact exists, and the measurements are very precise, and thus also statements can be made with regard to the required cooling measurement which can be carried out basing on these values much more purposefully than this has been possible according to the state of the art. Because of this information the medical staff, for example in a hospital, gets information about the condition of the patient and can determine suitable measurements with respect to the cooling without any problems. Nevertheless the patient remains movable to a high degree, and is not impeded in his or her freedom of movement by too heavy devices which cannot be carried, or devices by means of which he or she cannot move at all. The invention provides here a device by means of which these treatment measurements can be applied very purposefully and simply, in medical institutions as well as also during transporting sick people but also in the ambulant treatment.

In a preferred arrangement of the invention it is provided that at the device additionally at least one other measuring instrument or sensor for measuring physical parameters is provided, and thus the device can also measure surrounding parameters which cannot be influenced, even at the body. The basic aim of the suggestion according to the invention is the measurement and monitoring of the cooling efficiency or cooling performance or the actual physiological parameters in the area influenced by the device. For further monitoring it is an advantage here to correlate this information with additional surrounding parameters in order to get thus accordingly other information and knowledge.

Another aspect of the invention is given by the fact that other measuring instruments and/or sensors for collecting medical values of a human or an animal, such as blood pressure, electrocardiogram, blood, heart frequency/pulse, oxygen content of the blood and the like are provided. By means of these additional measuring instruments, microphones and/or sensors a complete monitoring of medical values is possible so that the efficiency of the device according to the invention is furthermore decisively increased. As the temperature of the body directly influences the values such as heart frequency/pulse, but also blood pressure and so on, the measurement of these values is thus an eminently important contribution to the monitoring of patients during cooling. By the employment of other measuring instruments it is possible to receive an endless amount of information about the user or patient, and this even over a longer period of time during which the device is carried.

After this period of time it is then again possible to get the information, for example via an evaluating unit which is connected extra for this purpose. However, it is also possible, to transmit this information permanently so that the attending doctor or the medical staff is able in each moment to call this information and to evaluate it accordingly. Of course, the invention also provides a device which transmits an automatic emergency call when there are deviations from the desired or normal values. Exactly in the respect to an emergency call or the telemedical monitoring the position of the patient is important in order to locate him or her via GPS, GPRS or other techniques when he or she is not responsive anymore or not able to make himself or herself heard. Besides, the sensor or the recognition of the spatial position is important in order to determine whether the patient suddenly fell down for health reasons, and in spatial respect may lay all of a sudden unconsciously on the floor. Sensors or devices which recognize this are known in the state of the art and can be integrated without any problems in the invention so that in the case of emergency quickly automatically help can be called. Of course, it is here possible, because of the equipping of the device according to the invention, to transmit also an exact position information (spatial position) of the patient. By means of that it is possible to discharge many patients from the very expensive treatment in the hospital and to allow them to live at home without stopping the necessary monitoring or the necessary medical cooling. This also saves enormous amounts of money in health care.

As a consequence of that the measuring instruments or sensors are or can be connected wireless and/or with connecting lines with a receiving, evaluating and/or display device. According to the invention it is an advantage here if at least one transmitting unit is provided which can communicate with a receiving station.

A modification of the invention provides here that the measuring instrument or the sensor is connected with the transmitting unit for a wireless or wired data transfer which transmits the measured values to a receiving station. Here several possibilities are comprised by the invention by means of which measuring instrument or sensor are connected with the transmitting unit, and the transmitting unit with the receiving station. Thus it is, for example, possible that measuring instrument or sensor are connected wired with the transmitting unit within the device, and a wireless data transfer to the receiving station is carried out, for example, via radio. Of course, also other ways of transmitting are comprised by the invention which may be solutions such as infra red, Bluetooth, GPS, GPRS or the like. The transmission of the values via radio waves is also comprised by the invention.

According to an advantageous modification of the invention it is provided that besides the measuring values, as described before, also positioning data with reference to the localization or the spatial position of the person carrying the device are generated by means provided at the device and are transmitted for evaluating purposes.

Therefore it is easily possible to locate where the respective person. It is also possible to transfer corresponding information to the person which, for example, tells the person to go to a certain place. It is also possible to transfer corresponding information with regard to the moisturizing with regard to the necessary cooling. In this respect the system is designed extremely universally.

According to the invention it is also provided that the means for transmitting the positioning data or the localization are combined with a speed sensor in order to detect whether the person carrying the device is in horizontal or vertical position, and, in particular, whether a change of position occurs normally or all of a sudden. Thus it can be determined whether the person moves, or whether, for example, the person goes to bed or gets in a sitting or horizontal position, and, in particular, the speed sensor can detect whether the change of position is the result of troubles in the movement processes of the person or the user, or whether it is a normal change of position. Thus it can be implicated, for example, whether the change of position has been caused by falling down or whether it has been a normal laying-down.

In another advantageous modification the invention suggests that at least one identification means is arranged at the device which can be allocated to the patient or the user or the animal. This can be, for example, a normal bar code which can be evaluated via a scanner. Of course, also other electronic ways are comprised by the invention by means of which the person or the animal carrying the device can be determined, such as for example by RSID systems.

As already mentioned it is, according to the invention, also provided that the evaluating and/or receiving or display device are arranged at the device itself. Furthermore, it is possible that the invention carries a data storage or the data storage is integrated in the device which stores the measured values in particular over a longer period of time, and which has a joint or interface for an evaluating unit. It is possible here to carry out long-term experiments and to give the person carrying the device for that term a freedom of movement as large as possible.

A device as described before is according to a development characterized in that at the device or the evaluating unit and/or receiving station a display and/or an input unit, such as for example a keyboard, a touch screen or the like is provided. By means of the display or the touch screen it is possible that the person carrying the device can then communicate with the system. At the same time it is possible to display also certain values to the carrier of the device.

After the end of the period of time for the examination or measuring then the information exchange between the data storage can be carried out at the device of the exterior evaluating unit. The evaluation can then, as usual, be done in computers or suitable evaluating apparatus. The patient or the person carrying the device does not have to be at the place of evaluation for that, but can wait for the evaluation without any problems separated from the information or evaluating units. An advantageous development of the invention suggests that the information exchange between the device and the exterior evaluating device is provided, in particular, bi-directional. By means of that it is possible to exchange information both ways. Thus it is accordingly provided that the values and information measured at the device are guided to the evaluating unit; however, vice versa also necessary information is transmitted and displayed from the evaluating unit to the device.

The invention also suggests that a data teletransmitting system can be connected to the device.

This is a very convenient modification and offers the possibility to give patients in nursing homes, wards or the like freedom of movement as large as possible, and to get nevertheless permanently information via the data teletransmitting system about the health situation and the location.

The device according to the invention is also characterized in that a power supply is provided internally or externally for the supply of sensors, transmitters and so on arranged at the device. This can be accumulators, batteries or the like. Of course, also wired systems are possible which, however, restricts the freedom of movement. For this reason accumulators will be preferred which, for example, can be charged again wired over night. It is also possible to provide suitable solar cells which provide the energy necessary for data collecting and transmitting. At the same time solar cells of this type can also provide the necessary energy to charge accumulators at the device.

It is another aspect of the invention that the supporting material is designed of natural and/or synthetic material or fibers, in particular as synthetic fleece of polyester, polypropylene or polyamide, for example as light and flexible filling material. In particular the light weight and the flexibility of the filling material are of special importance for the qualities of the device as, by means of that, it is possible to substitute the essentially too heavy devices of the state of the art completely by a new generation of light weight devices with a very high degree of flexibility and thus a high comfort in carrying. This comfort in carrying occurs despite the cooling effect which is the effect of the device, and thus provides a universal device which can be used very advantageously just in the field of medical applications. Of course, the invention is not restricted only to the use in the medical field but it can also be used conveniently in leisure time, sports or in normal areas, for example if very high outside temperatures make cooling of the body necessary or influence it positively. Then, for example, a vest or coat of the material of the device can be used and thus a cooling effect be reached.

The device is also characterized in that the super absorber (SAP=Super Absorbing Polymers) is formed of a net of flexible hydrophilic polymers of ionic as well as non-ionic nature which can absorb and bind and release again water liquids wherein during the releasing of the moisture by evaporating a cooling effect appears. The effect according to which during the releasing of moisture a cooling effect appears results in the advantages of the device according to the invention. The according to the invention convenient super absorber of a net of flexible, hydrophilic polymers is here in particular suited for the use in the device according to the invention. Here these SAP (Super Absorbing Polymers) of ionic as well as also non-ionic nature can be employed. Both modifications are comprised by the invention and thus equipped with the same advantageous effect.

The invention is characterized in that the super absorber is provided at or in the supporting material or the supporting fleece as gel or coating. Exactly the design with a coating has special advantages as the negative effects, as they occurred in the state of the art with a formed pocket in a carrier, are now completely avoided by the invention. It is now possible that because of the coating at or in the supporting fleece also a considerably larger surface is provided so that the effect can be increased altogether.

In another modification according to the invention it is provided that the supporting material is designed as paper inset, fabric layer or closed fabric pocket, on or in which the super absorber is arranged or imbedded as gel, in crystal form or as coating. When the super absorber is applied in crystalline form suitable water or moisture-permeable supporting materials are designed as closed fabric pockets in which the bulk-like, flowing crystalline super absorber is filled. The arrangement in the closed fabric pocket guarantees that the super absorber material is not spread uncontrolled in the entire device. Also a suitably designed fabric layer serves as supporting material according to the invention.

According to another advantageous modification the invention is characterized in that the super absorber is a super absorber binding water or liquid fast which can absorb water, for example, under the shower or faucet.

It is preferred here when the water absorption can be carried out within a period of 2 to 60 seconds, preferably 15 to 30 seconds. Because of the high velocity of the absorption of liquid or moisture by the device according to the invention the comfort of the device is further improved altogether. Now it is possible to “re-charge” the device accordingly in a very short period of time without the necessity of expensive techniques, as this has often been necessary in the state of the art. The invention is in its use very flexible and can, of course, also still be completed with a suitable water reservoir which, for example, during carrying the device within certain intervals guides the moisture to the device. It is, of course, also possible to design the complete device a bit more comfortably for example by arranging filling links such as hoses or connection pieces at the device which then can be connected to a water conduct.

The arrangement is also characterized in that the cover fabric is designed as cover. According to a modification the top and the bottom side can consist of different materials. Thus it can be provided, for example, according to the invention that the top side of the cover fabric or the cover consists of a moisture-permeable material and, in contrast to that, the bottom side of a moisture-impermeable material, of course, it is provided that the bottom side is nevertheless breathable so that the body can breath through the skin and this is not impeded by the bottom side. Suitable materials are known in the state of the art and have been combined according to the invention in the device in combination with the supporting fleece to the device according to the invention.

The invention also comprises a device which is designed multi-layered, wherein several layers of a cover fabric as well as also different cover fabrics form the layers. Furthermore it is provided here that between the single layers of a cover fabric supporting fleece is arranged. A multi-layered construction of this type further increases altogether the efficiency of the device. Additionally membranes can be built in which are either moisture-permeable or moisture-impermeable or steam-permeable or steam-impermeable.

According to the invention it turned out to be an advantage if at least a part of the cover fabric is designed as membrane and/or the membrane forms the cover fabric. By means of that other modifications can be realized for the device according to the invention which can be adapted to special medical purposes.

It is also an advantage if the cover fabric is formed of natural or synthetic fibers. Here a combination of natural or synthetic fibers is also comprised by the invention as the design of the device only with natural or only with synthetic fibers. The invention can, of course, also be used for other purposes. For this reason it is provided, according to a development of the invention, that as cover fabric fire-resistant or hardly inflammable material is provided. By means of that the device according to the invention can also be used as protective device in heat or fire situations. The device can also be used in heat zones as it may be necessary during steel production or in the chemical industry in certain rooms where high temperature prevails which, for example, a person should not enter without suitable protective gear.

It has furthermore been found to be an advantage if synthetic fleece layers coated with super absorber are arranged between several layers of the cover fabric. Furthermore it is an advantage if the synthetic fleece and the cover fabric are stitched tightly, glued or in another way connected with each other in such a way that the synthetic fleece is arranged in the cover fabric that it cannot slip. Such an embodiment can be realized, for example, in such a way that corresponding fabric lengths are pre-fabricated in the fabric already stitched to each other, and only then devices are produced in their different embodiments.

It is clear that the use of a synthetic fleece suggested according to the invention has the same effect as the use of the supporting material also suggested by the invention. The synthetic fleece is a special design of the supporting material suggested according to the invention.

In a development according to the invention it is provided that the supporting material, in particular supporting fleece, and the cover fabric are connected on at least one or a few points in such a way that the supporting material is arranged in the cover fabric in such a way that it cannot slip. Connecting or gluing over the entire surface of the supporting material with the cover fabric is not compulsorily decisive according to the invention, only a few connecting points are sufficient, if necessary even only one connecting point, this depends on the size of the supporting material arranged in the device.

Thus it is, according to the invention provided, for example, that the device has the form of blankets, clothing, covers, mats or the like. Furthermore it is convenient if the device is designed as vest, bandage, coat, pants, headgear, headband, cap, helmet, sleeping bag, cuff, or as bag or bag-like. It is a particular advantage if the device is or can be adapted to the shape of the body or the body part.

The invention also provides a medical cooling system comprising a device for cooling according to one of the preceding described modifications which furthermore comprises at least one measuring instrument or sensor each for measuring medical values, an evaluating and/or receiving or display device, a data teletransmitting system as well as an extern or intern power supply, and comprises furthermore an external evaluation unit. These components can be supplied individually so that, if desired, then suitable devices can be manufactured or combined depending on their field of use. Thus a universal system is at disposal which makes it possible to provide devices according to the invention either only as blanket or clothing, or as e.g. cuff, and to provide them with suitable measuring instruments or sensors which are just required for the respective purpose.

The invention also provides for the medical cooling system or for the parts of the system a storing or cooling container. This can be, for example, a climate cabinet, a refrigerator or a freezer, a refrigerator chest or freezer chest or the like. In this container, which, of course, can also be fitted to the form of the respective device, the device cannot only be stored but it can also be cooled for certain medical purposes. This is in particular necessary when the medical treatment requires a lower cooling temperature than this can be reached, for example, by evaporating cooling. In this respect here the advantages according to the invention can be combined excellently with normal cooling apparatus, and thus complete the medical cooling system according to the invention.

The invention furthermore comprises also a method for monitoring the cooling efficiency of a device as in particular described, wherein it is suggested according to the invention that in an evaluating unit the measuring values transmitted by the at least one measuring instrument or sensor are evaluated in such a way that the cooling efficiency is determined and the user of the device is informed about the cooling efficiency. Such a processing of the measuring values leads to a significant improvement of the comfort of the device according to the invention during use. The design of the algorithms for determining the cooling efficiency is here very variable and, of course, adapted to the respectively available measuring values. The method can expressly also be applied to other devices which serve the same purpose but do not have super absorber.

In a preferred modification it is provided here that, when the cooling efficiency is too low, the user is informed optically and/or acoustically and, if necessary, is requested to change the position and/or arrangement of the device at least for a short time. A significant removal of the body heat (the cooling effect is generated by that) is carried out by convection of the steam out of the device. Because of inconvenient position conditions or using conditions and so on it may be the case that the cooling efficiency decreases and this is below a defined threshold value and, cleverly before the user has a corresponding feeling of heat, he or she is optically and/or acoustically informed and also requested (alternatively) to change the position and/or the arrangement of the device at least for a short time, or, for example, to use another device. Namely, it has been found that the accumulation of steam in the vest can be removed, for example, by taking it down for a short time, turning it around and putting it on again. Such simple measurements can lead already again to a significant improvement of the cooling efficiency. The advantage of the invention is here in particular the fact that the device according to the invention without additional external cooling apparatus and without technical labour can be carried along in a simple manner, and, for example, be used in the working zone.

Of course, the invention also comprises, according to the method, all characteristics which have been described with regard to the invention, even if they have not been mentioned expressly in connection with the method. In this respect all characteristics described and disclosed in connection with the device have to be seen also in connection with the method as disclosed and can accordingly be combined. The same goes, of course, vice versa.

An improvement of the cooling effect is, of course, reached, when the entire device is, for example, pre-cooled or is operated with liquid which is accordingly cooled down. This can be reached by the corresponding use of cooled liquids or putting the device in suitable refrigerators. If necessary, here cooling can also mean a short-time freezing or freezing.

In the following the invention is described further by means of examples.

In the FIGURE:

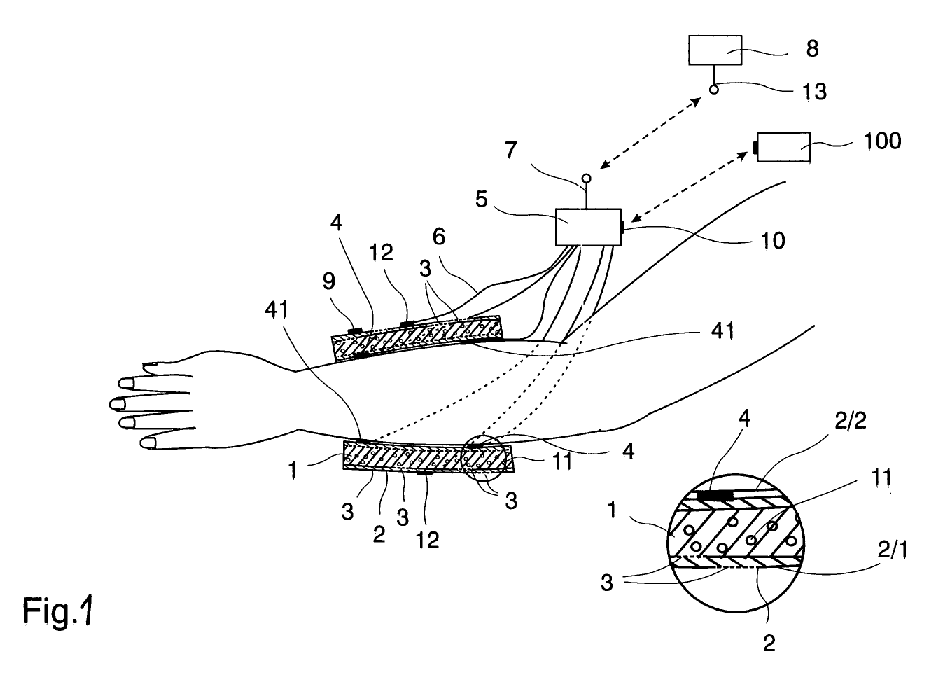

FIG. 1: a schematic demonstration of a single embodiment of the invention.

The single FIGURE shows an embodiment for a device according to the invention. The device is here shown essentially schematically and designed in the chosen modification as cuff. It is pointed out that according to the preceding description the design of the device according to the invention is possible in numerous modifications.

Thus it is, for example, possible to carry out the device as vest, as blanket, as headgear, as headband, as suit, as sleeping bag and so on. The parts of the invention, however, can be seen as shown in the embodiment also in other modifications which are not shown now.

The device according to the invention is, as already mentioned, designed as cuff wherein a supporting fleece 1 is arranged in a cover fabric 2 designed as cover. The cover fabric 2 has here a top side 2/1 and a bottom side 2/2. Because of the size of the FIGURE the design of a wall of the device according to the invention is shown on the bottom of the right hand side enlarged as circle section. This refers to the circle section in the normal FIGURE. Within the supporting fleece 1 super absorber parts 11 are arranged, schematically indicated, which, as suggested by the invention, have been arranged at the supporting fleece 1 by coating.

This embodiment makes it possible that a number of super absorber parts are integrated in the supporting fleece, and, nevertheless, the negative effects of the state of the art do not occur in such a way that dripping gel soils the clothing or that the pockets used in the state of the art in the supporting material have not been sufficiently sized to be able to absorb sufficient liquid, or that the supporting fleece has been arranged so inconveniently in the pockets that the liquid could not be absorbed accordingly or be released so that the cooling effect was not sufficient. It has been the result of the now chosen arrangement that the supporting fleece 1 has sufficient super absorber parts 11 so that a very good cooling could be noticed. On the side facing the body or the skin according to the invention either the measuring instruments 4 or sensors 41 are arranged. They are in the shown modification connected with a display unit 5 via connecting cables or connecting lines 6. Also other data are transmitted to the display device 5 from other measuring instruments provided at the device. Thus, for example, an identification means 12 is connected via a connecting line 6 with the display device 5. Furthermore an identification means 12 is provided which may be, for example, connected via radio or also via connecting cable with the display unit. It is, of course, also possible that the sensors and/or measuring instruments have their own transmitting devices in order to be able to communicate by radio with the display unit 5.

In the shown modification this is provided at least partly by cable. The display unit 5, however, has a transmitting unit 7 connected with the receiving unit 13. This receiving unit 13 is again arranged on a data teletransmitting system 8. The described modifications are, as mentioned in the beginning, only examples which do not restrict the invention in any way. It is rather shown by means of the chosen examples how the invention is constructed and how it works.

Reference number 3 shows permeable zones 3 on the top side 2/2 of the cover fabric 2 dash-dotted.

Through these permeable zones 3 moisture can enter in order to moisturize, for example, the super absorber 11 or the super absorber parts. The body heat makes the moisture of the super absorber or the supporting fleece evaporate, and it can escape to the outside through the permeable areas 3. According to the invention it is also suggested that the permeable areas 3 are provided, for example, on the top side 2/1 or in the top side 2/1 of the cover fabric 2. However, they can also be arranged on the top side and bottom side or even only on the bottom side. An interface 10, which can be connected or communicate with an evaluating unit 100, can be arranged on the display device 5. A modification is possible here where the patient carries the device during a certain period of time, and after this period has ended, the evaluating unit 100 takes over the data from the display unit which has, of course, a suitable storage. Of course here a direct communication, as indicated schematically by the double arrow, can take place via radio, infra red or the like. The invention is in no way restricted to a certain modification.

The invention is one more time summarized and shortly described, and the advantages which have to be reached according to the invention are again pointed out. The invention comprises essentially three parts, that is, first of all, a light and flexible foil material. This consists of a synthetic fleece of polyester, which is indicated here as supporting fleece 1. This supporting fleece 1 is coated with the super absorber (SAP) 11. The super absorber consists of nets of flexible, hydrophilic polymers which can be of ionic as well as of non-ionic nature. These are, as already described, able to absorb water liquids and to bind them, and are, however, also able to release this moisture again so that cooling is the result.

Now the invention solves the problems occurred negatively in the state of the art according to which cooling was necessary or according to which gel escaped, for example, from the cover and thus lead to soiling, or that, in the case that the cooling effect was not sufficient anymore, then another cooling of the device was necessary. The invention now provides a solution where neither water nor gel can escape. The binding of the water is carried out, as described further above, for example under the shower in seconds or under the faucet or in another way, for example by connecting to a connection piece in order to add moisture accordingly. Of course, also a water reservoir can be provided according to the invention which then supplies the necessary moisture when the cooling effect is no more sufficient. The cooling effect is, according to the invention, provided by evaporation cooling which again is effected by the body heat of the patient or user or the animal to be treated. According to that sufficient evaporation cooling or cooling effect is provided for medical applications by the device according to the invention.

The second part of the invention consists of a cover fabric enveloping the fleece which is already available on the market in many colours and variations of different manufacturers. It is, of course, provided here that the top and bottom parts of the cover or also other parts can be different. According to that a cover fabric can be provided 1 to x times. Of course, it is also possible to use suitably fire-resistant or hardly inflammable materials. In the same it is, of course, also provided that the cover fabric is made of different materials. The material can here be provided as yardware. Of course, it can also be in the form of blankets or mats in different sizes, and also as clothing for medical cooling. As already mentioned the cover according to the invention has one or more places or sides which are air and/or steam-permeable in order to guarantee absorption and evaporation of the bound water so that the cooling effect occurs.

The third aspect of the invention is described in the following:

The finished product in the form of blankets or mats in different sizes or in the form of clothing, headgear or in the form of pads is equipped with the telemedical measuring instruments 4 or sensors 41 in such a way that the control of the medical cooling is possible in the area temperature measuring but also in the area of measuring other body functions in the medical field. In order to guarantee freedom of movement in most of the applications this measurement can be transmitted also wireless, according to an embodiment of the invention, or, as already also described, it can be stored for later evaluation. The data can also be combined with suitable evaluating systems or with monitoring system which are used in the field of medicine. Also a data teletransmission or tele-servicing and transmission is comprised by the invention. The measurements carried out at the device according to the invention and provided by the system according to the invention, are, among others, measurement of temperature, measurement of blood pressure, measurement of pulse/heart frequency, ECG, measurement of oxygen content of the blood, localization of the patient, spatial position of the patient or identification of the patient. The measuring values are evaluated directly at the invention and displayed, or they are requested by a tele-system and stored there or evaluated. Thus short-time or long-time measurements can be carried out without the person to be connected to the apparatus. The full freedom of movement remains which has significant advantages in the field of living quality, and makes it also possible that the concerned person remains, for example, able to work. In addition to that these systems can also be connected in the area of telemedicine to data teletransmission systems which saves stays in the hospital and visits to doctors, and thus also reduces expenses in health care.

Summarized the following advantages are the result of the invention.

The invention is optimally suited for cooling in the field of medicine as it complies with the following requirements:
1. Medical cooling by physical evaporation of the absorbed water;
2. Very light material and thus easy transport or easy storing and use for staff; therefore, first of all, no stress for patients and affected persons;
3. Very flexible material so that a flexible use for many product applications and optimal cooling effect is possible by use close to the body.
4. Fast, controllable water absorption in seconds up to ten or twenty times of its weight without releasing it again by mechanic pressure; by means of that there is no wetness which wets the affected persons or prevents telemedical applications and medical measurements;
5. The device according to the invention can be recycled so that the product is environment friendly;
6. No additional cooling energy is required, this also serves for improving the CO₂ balance and is convenient accordingly for the climate balance;
7. It is safe for men and nature;
8. Is equipped or can be equipped according to the progress of medicine with up-to-date medical and telemedical control, monitoring and evaluating methods;
and
9. Self monitoring of the cooling efficiency of a self-sufficient device working passively.

Of course, different fleece materials with different thickness as well as ability to bind water can be produced and used depending on the purpose. By a higher thickness of the material or higher quantity of the super absorber coating of the fleece the level of the ability of binding water increases. Of course, thus also the weight of the fleece per square meter increases, however, this occurs only to a small degree and thus has hardly any influence on the handling.

The claims filed with the application now and to be filed later on are attempted formulations without prejudice for obtaining a broader protection.

If here, on closer examination, in particular also of the relevant prior art, it turns out that one or the other feature may be convenient for the object of the invention, however, not decisively important, of course, already now a formulation is striven for which does not contain anymore such a feature, in particular in the main claim.

Furthermore it has to be taken into consideration that the embodiments and modifications of the invention described in the different examples and shown in the figures can be combined with each other in any way. Here single or several characteristics can be exchanged in any way. These combinations of characteristics are also disclosed.

References in the sub-claims relate to the further design of the matter of the main claim through the characteristics of the respective sub-claim. These are, however, not to be understood as a waiver of independent, subjective protection of the matter for the characteristics of the referred sub-claims.

Characteristics only disclosed in the description so far may now, in the course of proceedings, be claimed as being of inventive relevance, for example to distinguish from the state of the art.

Characteristics only disclosed in the description or even single characteristics of claims comprising a number of characteristics may be taken over in the first claim at any time to distinguish from the state of the art, and this is even if such characteristics have been mentioned in connection with other characteristics, and achieve particularly convenient results in connection with other characteristics, respectively.

1-52. (canceled)

53. A device for cooling, in particular for cooling bodies or body parts of a human or an animal, comprising at least one supporting material with super absorber (SAP) which is able to absorb, bind and release again moisture or liquid, wherein a cooling effect occurs by evaporating when the moisture is released, wherein the supporting material is enveloped by a cover fabric and the cover fabric has at least one moisture permeable or air and water steam permeable area, characterized in that at least one measuring instrument and/or sensor for measuring physical measuring values is provided at or in the device.

54. The device according to claim 53, characterized in that by means of the measuring instrument and/or sensor determining and/or monitoring the cooling efficiency is carried out and/or as measuring instrument or sensor at least one thermometer or thermal sensor is provided and/or as measuring instrument or sensor, in particular for determining the cooling efficiency, a measurement of the temperature difference is provided at the device, in particular at the supporting material.

55. The device according to claim 53, characterized in that as measuring instrument or sensor, in particular for determining the cooling efficiency, an air moisture sensor or a measurement of the difference of the air moisture is provided at the device, in particular at the supporting material and/or the measuring instrument or the sensor, in particular for determining the cooling efficiency, measures the partial pressure of the water steam, in particular the difference of the partial pressure of the water steam, at the device, in particular at the supporting material and/or as measuring instrument or sensor, in particular for determining the cooling efficiency, a moisture charging sensor for determining the liquid bound in the supporting material is provided at the device, in particular at the supporting material.

56. The device according to claim 53, characterized in that as measuring instrument or sensor, in particular for determining the cooling efficiency, a climate measuring apparatus is provided at the device, in particular at the supporting material and/or the cooling efficiency is determined by measuring the difference of the measure values either with regard to the measuring location and/or with regard to the measuring moment.

57. The device according to claim 53, characterized in that as measuring instrument a thermometer is designed as liquid crystal thermometer and/or for determining the cooling efficiency the measuring instrument or the sensor is equipped or connected with an evaluating unit and/or a display for the cooling efficiency is provided showing the information determined by the evaluating unit.

58. The device according to claim 53, characterized in that the measuring instrument or the sensor is arranged in or at the supporting material and/or the measuring instrument or the sensor is arranged on the side of the supporting material facing and/or opposite the human or animal and/or a protective pocket or protective flap at the device for the measuring instrument or the sensor is provided.

59. The device according to claim 53, characterized in that the measuring instrument or the sensor is arranged on the side of the cover fabric or sheathing facing the body, preferably in such a way that the measuring instrument or sensor comes into direct body or skin contact when the device is put on and/or at the device additionally at least one other measuring instrument or sensor is provided for measuring physical parameters, and by means of that not-influenced environmental parameters are measured by the device, also at the body and/or other measuring instruments and/or sensors are provided for collecting medical values of a human or an animal, such as blood pressure, pulse/heart frequency, oxygen content of the blood or the like.

60. The device according to claim 53, characterized in that the measuring instruments or sensors are or can be connected wireless and/or with connecting lines with a receiving or evaluating and/or display device and/or at least one transmitting unit is provided and/or the measuring instrument or the sensor is connected with the transmitting unit for a wireless or wired data transmission transmitting the measuring values to a receiving station.

61. The device according to claim 53, characterized in that the measuring instrument or the sensor is connected with the transmitting unit and as transmitting unit and/or receiving station a GPS (Global Positioning System) or GPRS (General Packet Radio Service) unit is provided and/or besides the measuring values also positioning data with regard to the localization or spatial position of the human carrying the device can be generated by means provided at the device and can be transmitted for evaluating purposes.

62. The device according to claim 53, characterized in that the device can be generated by means provided at the device and the means for transmitting the positioning data or localization are combined with a speed sensor to detect whether the human carrying the device is in horizontal or vertical position, and in particular whether a change of positions occurs normally or all of a sudden.

63. The device according to claim 53, characterized in that at least one identification means is arranged at the device which can be allocated to a patient and/or a evaluating and/or receiving or display device is arranged at the device and/or at the device or at the evaluating and/or receiving station a display and/or an input unit, such as for example a keyboard, a touch screen or the like is provided.

64. The device according to claim 53, characterized in that a data storage is provided at the device storing the measuring values, in particular for a longer period of time and having a joint or an interface for an exterior evaluating unit and/or the information exchange between the device and exterior evaluating unit is provided in particular bi-directional and/or a data tele transmission system can be connected with the device.

65. The device according to claim 53, characterized in that an power supply is provided internally or externally for supplying sensors, transmitters and so on arranged at the device and/or the supporting material is designed as supporting fleece of natural and/or synthetic material or fibers, in particular as synthetic fleece of polypropylene, polyamide, polyester, for example as light and flexible filling material.

66. The device according to claim 53, characterized in that the super absorber (SAP=super-absorbing polymers) is formed of a net of flexible hydrophilic polymers of ionic as well as also non-ionic nature, which is able to absorb and bind and release again watery liquids, wherein a cooling effect by evaporating occurs when the moisture is released and/or the super absorber is provided as gel, in crystal form or coating at or in the supporting material, in particular at the supporting fleece and/or the supporting material is designed as paper inset or closed fabric pocket on or in which the super absorber is arranged or bedded in as gel, in crystal form or coating.

67. The device according to claim 53, characterized in that the super absorber is a super absorber binding water or liquid fast which can absorb water, for example under the shower or the faucet, in particular in a period of time of 2 to 60 seconds, preferably in 15 to 30 seconds and/or the cover fabric is designed as sheathing and/or the top side—and the bottom side of the cover fabric consist of different materials.

68. The device according to claim 53, characterized in that the device is designed with multiple layers, wherein several layers of a cover fabric as well as also different cover fabrics form the layers and/or at least a part of the cover fabric is designed as membrane and/or the membrane forms the cover fabric and/or is connected with a membrane and/or the cover fabric is formed by natural and/or synthetic fibers and/or as cover fabric fireproof or hardly inflammable material is provided.

69. The device according to claim 53, characterized in that the supporting material coated by the super absorber, in particular the supporting fleece, is arranged between several layers of the cover fabric and/or the supporting material, in particular the supporting fleece and the cover fabric are tightly stitched, glued or connected to each other in another way in such a way that the supporting material is arranged in the cover fabric in a non-slipping way and/or the supporting material, in particular the supporting fleece and the cover fabric, are connected to each other at least at one or a few points in such a way that the supporting material is arranged in the cover fabric in a non-slipping way.

70. The device according to claim 53, characterized in that the device has the shape of blankets, clothing, covers, mats, the shape of pads or the like and/or the device is designed as vest, bandage, coat, pants, headgear, head band, cap, helmet, sleeping bag, cuff, as bag or bag-like and/or the device is or can be fitted to the shape of the body or a body part.

71. The medical cooling system comprising a device for cooling, in particular for cooling bodies or body parts of a human or an animal, comprising at least one supporting material with super absorber (SAP) which is able to absorb, bind and release again moisture or liquid, wherein a cooling effect occurs by evaporating when the moisture is released, wherein the supporting material is enveloped by a cover fabric and the cover fabric has at least one moisture permeable or air and water steam permeable area, at least one measuring instrument or sensor each for measuring medical values, an evaluating and/or receiving or display device, a data teletransmitting system, an external or internal power supply as well as an external evaluating unit.

72. The medical cooling system according to claim 71, characterized in that for the system or the system parts a storing and/or cooling container such as for example a climatic cabinet, refrigerator or freezer, a refrigerator chest or freezer chest or the like is provided where the device can be stored and/or cooled.

73. The method for monitoring the cool efficiency, in particular a device for cooling, in particular for cooling bodies or body parts of a human or an animal, comprising at least one supporting material with super absorber (SAP) which is able to absorb, bind and release again moisture or liquid, wherein a cooling effect occurs by evaporating when the moisture is released, wherein the supporting material is enveloped by a cover fabric and the cover fabric has at least one moisture permeable or air and water steam permeable area, wherein at least one measuring instrument and/or sensor for measuring physical measuring values is provided at or in the device, characterized in that in an evaluating unit the measuring values transmitted by the at least one measuring instrument or sensor are evaluated in such a way that the cooling efficiency is determined and the user of the device is informed of the cooling efficiency.

74. The method according to the preceding claim 73, characterized in that when the cooling efficiency is too low the user is informed optically or acoustically, and is, if necessary, requested to change the position and/or the arrangement of the device at least for a short period of time.
